SEQUENCE LISTING (4 sequences)

SEQ ID NO: 1 - protein, 151 aa, Homo sapiens
IIDEWRKKTDDLAAELDGAQRDLRNTSTDLFKAKNAQEELAEVVEGLRRENKSLSQEIKDLTDQLGEGGRSVHEMQKIIRRLEIEKEELQHALDEAEAALEAEESKVLRAQVEVSQIRSEIEKRIQEKEEEFENTRKNHARALESMQASLE

SEQ ID NO: 2 - protein, 16 aa, Homo sapiens
LQHALDEAEAALEAEE

SEQ ID NO: 3 - protein, 16 aa, Homo sapiens
LQHALDEAEAALEAEE

SEQ ID NO: 4 - protein, 20 aa, Homo sapiens
EELQHALDEAEAALEAEESK

(19) United States
(12) Patent Application Publication
(10) Pub. No.: US 2019/0018928 A1
(43) Pub. Date: Jan. 17, 2019

(54) Methods for Mass Spectrometry-Based Structure Determination of Biomacromolecules

(71) Applicants: VITO NV, Mol (BE); Universiteit Antwerpen, Antwerpen (BE)

(72) Inventors: Dirk Valkenborg, Mol (BE); Jef Hooyberghs, Mol (BE); Kris Laukens, Wilrijk (BE)

(73) Assignees: VITO NV, Mol (BE); Universiteit Antwerpen, Antwerpe (BE)

(21) Appl. No.: 16/066,846

(22) PCT Filed: Dec. 30, 2016
(86) PCT No.: PCT/EP2016/082907

§ 371 Date: Jun. 28, 2018

(30) Foreign Application Priority Data
Dec. 30, 2015 (EP) EP15203056.5

(51) Int. Cl. G06F 19/24; G01N 33/68; C12Q 1/6872; G06F 19/12; H01J 49/00

(52) U.S. Cl. CPC G06F 19/24; G01N 33/6848; H01J 49/0036; G06F 19/12; C12Q 1/6872

(57) ABSTRACT

Methods and tools for determining the structure of biomacromolecules such as proteins in a sample using mass spectrometry. More particularly the methods allow determining the presence of a biomacromolecule of an organism in a sample by comparing an observed mass spectrum of the sample with a theoretical fragment ion spectrum comprising theoretical fragment ion masses.

The present application claims priority from International Patent Application No. PCT/EP2016/082907 filed on Dec. 30, 2016, which claims priority from European Patent Application No. EP 15203056.5 filed on Dec. 30, 2015, the disclosures of which are incorporated herein by reference in their entirety.

### FIELD OF THE INVENTION

The invention provides methods and tools for determining the structure of biomacromolecules such as proteins in a sample using mass spectrometry.

Large scale proteomics gained much attention once it became possible to acquire a large number of spectra (Michalski, A. et al., 2011, Mol. Cell. Proteomics MCP 10, M111.011015), and to identify a reasonable proportion of the resulting spectra in an automated fashion (Eng et al. (1994) J. Am. Soc. Mass Spectrom. 5, 976-989). Nevertheless, to this day between 33% to 50% of the acquired spectra remain unidentified. Essentially all proteomic work to date has been done at the peptide level by first enzymatically digesting the proteins to shorter peptides with more homogenous and tractable chemical characteristics. These are then introduced (usually following some form of physical/chemical separation) into a mass-spectrometer where spectra are acquired. The work of generating peptide spectral matches (PSMs) is then taken on by one of three styles of identification algorithms: (1) database searching tools compare the observed spectra to predicted spectra based on in-silico digestion of the target organism's proteome, (2) Tag-based de-novo techniques attempt to identify the peptide by direct interpretation of the spectrum followed by comparison to a database of predicted peptides and (3) the use of spectral libraries to identify the peptide by direct comparison of the query spectrum against a library of empirically derived reference spectra (usually referred to as a spectral library).

All of these approaches (including spectral libraries) rely heavily on the value of the query peptide's total mass (prior to tandem MS-fragmentation) typically referred to as the precursor mass. This is because the overall mass allows the algorithm to exclude a large proportion of the possible candidate peptides. The list of candidate peptides is itself kept to a minimum by the use of enzymes with sufficient specificity to produce only a relatively limited number of peptides per protein. Users typically ignore or severely limit the set of possible post translational modifications which the protein may have undergone (in-vivo) or the peptide itself may have acquired (in-vitro). The common thread to all peptide-centric approaches to proteomics is the requirement to: (1) know the proteome (sequence) of the target organism, (2) rely on a highly specific enzyme digestion process, and (3) filter candidate peptides by the precursor mass which yielded the observed fragment peaks. Furthermore, these techniques all assume that observed peptide spectra will be matched with predicted (in-silico derived) peptide spectra.

An alternative approach to mass-spec based proteomics is to avoid the digestion step and attempt to identify the protein by direct fragmentation of the entire protein in the mass-spectrometer (Tran, J. C. et al., 2011, Nature 480, 254-258). This presents, in principle, a very strong benefit in terms of characterization of the so-called proteoform (the overall combination of post translational modifications (PTMs) co-occurring on the protein—essentially its overall state), but the cost is a drastic limitation in the throughput of the technique as well as a strong increase in the complexity of the resulting spectra. This is because the approach is still in its infancy as evidenced by the relatively few published studies involving anything approaching high-throughput in the bottom-up realm. Nevertheless, computational techniques have been developed to match spectra generated from the entire protein (especially after a pre-processing step called de-convolution) to its putative source (see e.g. Cannon, J. et al. 2010, J. Proteome Res. 9, 3886-3890. These are termed “PRSMs” to distinguish them from matches to individual peptides. Here again the algorithms typically rely on a knowledge of the overall mass of the precursor and they are all premised on the notion that a comparison is made between observed protein spectra and predicted (in silico derived) protein spectra.

Several optimization approaches have been described in the art, for instance, the application of a precursor mass filtering (Geer, L. Y. et al. 2004, J. J Proteome Res. 3(5), 958-964) and the matching of the observed peptide fragments thereafter on a theoretical spectrum of merged B- and Y-ions. Other examples include, MASCOT, SEQUEST, X!TANDEM, SPECTRUM MILL, Andromeda, MS Amananda, CRUX. Additionally, a clustering of the observed fragment and the next matching can be performed (Frank, A. M. et al. 2008, J. Proteome Res. 7(1), 113-122) to improve the running time of the analysis in comparison with a database search. Alternatively, a search can be performed for spectral similarities between observed and identified spectra and spectra that are not identified (Wilhelm, T. et al. 2014, J. J Proteome Res. 13(9), 4002-4011), the core of which relies on a mathematical convolution of the spectral data. Each of these approaches, however, fail to address one or more problems of the art. Similar challenges exist in the determination of the structure of other biomacromolecules such as nucleic acids and polysaccharides.

There is a need in the art for improved proteomics, genomics, transcriptomics, and glycomics methods.

### SUMMARY OF THE INVENTION

The methods provided herein address one or more drawbacks of the methods of the prior art.

The inventors have found a way to leverage accurate mass instrumentation with a theoretical fragment ion spectrum to be matched against an observed mass spectrum. Applied to proteins, a PRSM-style predicted spectrum can be matched against a peptide-scale spectrum. The biomacromolecules need not be in any way limited in terms of the digestion that produced them. In the context of proteins, an important advantage of the present methods is that the peptides need not be tryptic, they can even be endogenous, and there is no limitation on the PTMs which they may be manifesting, the fragmentation mechanism used or even the purity of the spectra. The methods provided herein are based on the ability to quickly recognize a series of peaks as having been produced by an arbitrary subsequence of the full proteome. This can be done with high specificity and sensitivity thanks to the accuracy of modern mass spectrometers. In particular embodiments, the speed comes from the application of infinite accuracy spectral convolution followed by a single-pass clustering technique. The result is an assignment of peaks from original, raw scan to a region of a specific biomacromolecule (so called hot-spot). The assignment may be reported with an associated p-value as the distribution of convolution scores follow a Poisson distribution. Interestingly this method can equally be applied to other biomacromolecules, such as sugars or nucleotide sequences. In some embodiments, the only pieces of input required in methods provided herein are: (a) raw spectra to be analyzed, i.e. observed mass spectra which stem from one or more mass spectroscopy experiments, (b) the biological sequence which is looked for, e.g. a FASTA file with the proteome/genome/transcriptome or glycome of the target organism, and (c) a user-defined threshold (or clustering parameter) which corresponds to the accuracy of the mass spectrometer which was used to obtain the observed mass spectra of step (a).

More particularly, the invention provides computer-implemented methods for determining the presence of a biomacromolecule of an organism in a sample. The methods of the invention comprise the step of comparing an observed mass spectrum of the sample with a theoretical fragment ion spectrum comprising theoretical fragment ion masses of the biomacromolecule of interest.

The methods of the present invention can be applied to different types of biomacromolecules such as to proteins, nucleic acids, and polysaccharides.

In particular embodiments, the biomacromolecule is a protein.

The methods of the present invention can be applied to different types of mass spectroscopy techniques and the embodiments described herein serve as a proof of concept. Additionally, the methods can also be combined with other separation techniques (e.g. chromatography, mobility) and data tools (e.g. analysis, interpretation, representation, characterization) known in the art.

In some embodiments, the methods comprise the step of obtaining a mass spectrum of the sample thereby obtaining a set of query peaks; subtracting the m/z value (i.e. the mass-charge ratio) of every query peak from the theoretical fragment ion masses of the macromolecules (for the organism of interest); clustering and scoring the resulting differences, thereby obtaining scores representative of the likelyhood of the presence of a specific biomacromolecule; and assigning the spectrum to a specific biomacromolecule, based on said scores, thereby identifying the presence of said biomacromolecules in said sample.

In particular embodiments, where the biomacromolecule is a protein, said method comprises the steps of:
obtaining an observed mass spectrum of the sample thereby obtaining a set of query peaks;
subtracting the m/z value of every query peak from the theoretical fragment ion masses of the target proteome of said organism obtained by determining theoretical fragment ion spectra comprising theoretical fragment ion masses for the protein sequences of said target proteome;
clustering and scoring the resulting differences, thereby obtaining scores representative of the likelyhood of the presence of a specific protein in said sample; and
assigning the observed spectrum to a protein of said proteome, based on said scores, thereby identifying the presence of said protein in said sample.

In particular embodiments, the observed mass spectrum is obtained by tandem mass spectroscopy.

In particular embodiments, the methods comprise the step of obtaining a tandem mass spectrum of the sample thereby obtaining a set of query peaks; subtracting the m/z value (i.e. the mass-charge ratio) of every query peak from the theoretical fragment ion masses of the macromolecules (for the organism of interest); clustering and scoring the resulting differences, thereby obtaining scores representative of the likelyhood of the presence of a specific biomacromolecule; and assigning the spectrum to a specific biomacromolecule, based on said scores, thereby identifying the presence of said biomacromolecules in said sample.

In particular embodiments, where the biomacromolecule is a protein, said method comprises the steps of:
obtaining a tandem mass spectrum of the sample thereby obtaining a set of query peaks;
subtracting the m/z value of every query peak from the theoretical fragment ion masses of the target proteome of said organism obtained by determining theoretical fragment ion spectra comprising theoretical fragment ion masses for the protein sequences of said target proteome;
clustering and scoring the resulting differences, thereby obtaining scores representative of the likelyhood of the presence of a specific protein in said sample; and
respectively assigning the spectrum to a protein of said proteome, based on said scores, thereby identifying the presence of said protein in said sample.

In some embodiments, said theoretical fragment ion spectra are obtained by assuming that at least 25% of all possible ions, more preferably at least 75% of all possible ions, most preferably by assuming all possible ions are generated from the sequences in said proteome. Similar analyses can be made for other biomacromolecules such as for nucleotide sequences based theoretical fragment masses of the genome or for polysaccharides based on the theoretical fragment masses of the glycome.

In some embodiments, the method comprises generating theoretical ion masses for the target proteome, genome or glycome and computing error intervals for every fragment ion mass based on the error tolerance of the mass spectrometry instrumentation.

In some embodiments, the method comprises selecting a theoretical fragment ion spectrum i corresponding to a given protein, nucleotide sequence or polysaccharide and comparing it to an observed fragment ion spectrum j.

In some embodiments, the method comprises, for every observed fragment mass, selecting a mass value py from observed fragment spectrum j, selecting a mass value mx from theoretical fragment spectrum i, aligning the observed spectrum to the theoretical spectrum by
computing a mass shift τxy=mx−py; and
adjusting the observed fragment ion masses by adding τxy, such that the new mass of peak py now equals mx.

In some embodiments, the method further comprises searching for a pattern and scoring said pattern by a method comprising the steps of:
a) computing the number of fragment ion masses that coincide with the adjusted masses of the observed fragment ion spectrum given the precomputed error tolerance intervals (count_{xy});
b) computing the sum of the intensities of the observed fragment ion masses coinciding with theoretical fragment ion masses (sum_{xy}).

In some embodiments, the method further comprises modeling the distribution of the number of coinciding fragment ions by a Poisson model and generating for each location a p-value for the probability of a match between an observed fragment ion spectrum and a (part of) a theoretical ion spectrum, and optionally correlating the local score distribution for additional confidence, wherein positions with a p-value smaller than a pre-determined significance level are regarded as statistically significant.

In some embodiments, the method further comprises annotating the observed fragment spectrum to show which peaks were matched by the theoretical ion fragments and updating the sequence to show which subsequence had observed matching ion fragments.

In some embodiments, the theoretical fragment ion masses are adjusted by assuming a charge state z: the “theoretical fragment ion masses” are updated to “(theoretical fragment masses)/z”.

In some embodiments, the sample comprises more than one biomacromolecule from said organism.

Further provided is a data-processing system comprising means configured for carrying out a method provided herein.

Further provided is a computer program product having instructions which when executed by a computing device or system cause the computing device or system to perform a method provided herein.

### BRIEF DESCRIPTION OF THE DRAWINGS

The following description of the figures of specific embodiments of the invention is merely exemplary in nature and is not intended to limit the present teachings, their application or uses.

FIG. 1 shows a schematic illustration of how the methods and systems according to particular embodiments of the present invention investigate peptide spectra data against a protein sequence reference database according to a particular embodiment of the invention.

FIG. 2 shows a schematic illustration of protein character strings converted into numeric sequences by converting every letter, to the mass of the predicted ion for the associated Amino Acid letter code. The resulting mass is appended onto a running total of ion masses, effectively resulting in a synthetic spectrum against which comparisons of real spectra can be made.

FIG. 3 shows exemplary scores for the extent to which an observed mass spectrum corresponds to a particular (part of) a theoretical mass spectrum. In particular, the figure shows how a given empirical (i.e. observed) spectrum against every possible theoretical start-stop position (i.e. peptide fragment) in every possible protein of the reference proteome.

FIG. 4 shows the graphical representation of results obtained by the methods and systems according to particular embodiments of the invention includes: (a) annotation of observed ions with their matched pseudo-ions and (b) scoring of the protein sequences with the counts and/or intensities of their matching observed peaks.

FIG. 5 shows an exemplary observed CID-spectrum of peptidic origin according to an embodiment of the invention

FIG. 6 shows matching pseudo-ion counts for both the b-ion series (a) and the y-ion series (b) according to an embodiment of the invention.

FIG. 7 shows a Poisson model used in p-value estimation along with observed match to empirical count distribution according to an embodiment of the invention.

Throughout the figures, the following numbering is adhered to: 1—spectrum collection; 2—spectral matching; 3—spectral assignment; 4—protein database; 5—theoretical fragment ion spectra; 6—sequence coverage protein i.

### DETAILED DESCRIPTION OF EMBODIMENTS

It is to be understood that the figures and descriptions of the present invention have been simplified to illustrate elements that are relevant for a clear understanding of the present invention, while eliminating, for purposes of clarity, many other elements which are conventional in this art. Those of ordinary skill in the art will recognize that other elements are desirable for implementing the present invention. However, because such elements are well known in the art, and because they do not facilitate a better understanding of the present invention, a discussion of such elements is not provided herein.

The present invention will now be described in detail on the basis of exemplary embodiments.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional elements or method steps. The terms “comprising”, “comprises” and “comprised of” when referring to elements or method steps cited herein also include embodiments which “consist of” said elements or method steps cited herein.

Furthermore, the terms first, second, third and the like as used herein are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

The recitation of numerical ranges by endpoints includes all numbers and fractions subsumed within the respective ranges, as well as the recited endpoints.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

In the present disclosure, the algorithms provided herein are mostly described in the context of protein sequencing, but it is to be understood that the algorithms are by no means limited to protein sequencing as such. The underlying principles are equally applicable to determining the structure of other biomacromolecules such as nucleic acids and polysaccharides. In particular, DNA, proteins, peptides, carbohydrates, like sugars and fibres are bio-polymers that are composed of a well-defined alphabet. In the case of DNA this is {ACGT} and we have a 20-letter alphabet in the case of proteins and peptides. Any fragmentation technique for mass spectroscopy (MS), e.g. tandem MS, that produces reproducible fragment ions for the biopolymers will generate data that is compatible with the present methods, which allows relating en observed fragment pattern to a text pattern. The present disclosure focuses on peptide to protein mapping, but a skilled person understands that the same principles also hold for e.g. gene to chromosome matching.

The present inventors have identified a new way which allows for the identification of biomacromolecules in a sample based on the matching of peptide spectra. Generally, in some embodiments, the method comprises the step of comparing an observed mass spectrum of a sample with a theoretical fragment ion spectrum.

In some embodiments, the observed spectrum is generated from a sample of a target organism. Also, accurate-mass clustering may be applied to said spectrum, for example by taking into account precomputed error intervals, as is detailed below. The observed spectra may correspond to a single peptide, or they may be chimeric or mixed (i.e. comprising ions from more than one peptide).

The theoretical fragment ion spectrum typically comprises theoretical fragment ion masses and theoretical fragment ion series.

In some embodiments, the observed mass spectrum is compared to the theoretical ion-series of every intact biomacromolecule in the set of the particular biomacromolecules present in a target organism.

When the present methods are applied to proteins, the method preferably involves comparing an observed mass spectrum with a theoretical fragment ion spectrum which comprises predicted ion series of every intact protein in the target organism's proteome. Preferably, the theoretical fragment ion spectrum is clustered before the comparison, wherein the clustering preferably takes into account the accuracy of the mass spectrometer by which the observed mass spectrum is obtained. This clustering step is explained in detail below. The result is an assignment of peaks from the spectra to sub-sequences in the protein database.

When applied to proteins, methods according to the invention typically require only an observed mass spectrum and a FASTA file of the reference proteome. In particular, any information about precursor masses, digestion protocols, fragmentation techniques, expected PTMs (post-translational modifications) or mutations is typically not required.

The methods allow for the interpretation of mass spectrometry-generated fragment ion spectra of amino acid sequences based on a search against a protein database that does not depend on user defined parameters. In the context of protein sequencing, the only two parameters are inferred from the experimental set-up and include the protein database (defined by the organism under scrutiny) and the mass accuracy (defined by the mass spectrometer).

The invention thus provides a computer-implemented method for determining the presence of a biomacromolecule of an organism in a sample wherein the method comprises the step of comparing an observed mass spectrum of the sample with a theoretical fragment ion spectrum comprising theoretical fragment ion masses. In some embodiments, the comparison involves spectral convolution of the observed mass spectrum with the theoretical fragment ion spectrum. Preferably, the biomacromolecule is selected from the list consisting of proteins, nucleic acids, and polysaccharides. When the biomacromolecule is a protein, the method may comprise comparing the obtained mass spectrum of the sample with the predicted a/x, b/y, and/or c/z series of the target proteome.

More particularly, the biomacromolecule is a protein or a nucleic acid and the method comprises the steps of:
obtaining a tandem mass spectrum of the sample thereby obtaining a set of query peaks;
subtracting the m/z value of every query peak from the theoretical fragment ion masses of the target proteome or genome of said organism obtained by determining theoretical fragment ion spectra comprising theoretical fragment ion masses for the protein or nucleic acid sequences of said target proteome or genome, respectively;
clustering and scoring the resulting differences, thereby obtaining scores representative of the likelyhood of the presence of a specific protein or gene in said sample; and
respectively assigning the spectrum to a protein of said proteome, or to a gene of said genome, based on said scores, thereby identifying the presence of said protein or gene in said sample.

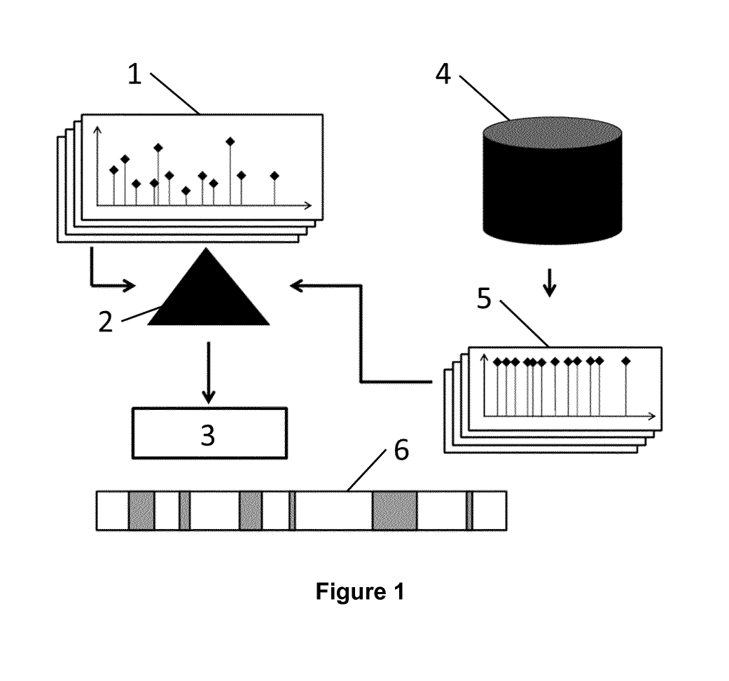

The principle is illustrated in FIG. 1 for the case in which the biomacromolecule is a protein. As described herein, tandem mass spectrometry refers to a particular approach wherein distinct ions of interest are selected based on their m/z value from the first round of mass spectrometry and are fragmented by a number of methods of dissociation (e.g. collisions with (high energy) inert gas, electron-transfer, electron-capture, etc.). These fragments are then separated based on their individual m/z ratios in a second round of mass spectrometry.

In a particular, where the biomacromolecule is a protein, said method comprises the steps of:
obtaining an observed mass spectrum of the sample thereby obtaining a set of query peaks;
subtracting the m/z value of every query peak from the theoretical fragment ion masses of the target proteome of said organism obtained by determining theoretical fragment ion spectra comprising theoretical fragment ion masses for the protein sequences of said target proteome;
clustering and scoring the resulting differences, thereby obtaining scores representative of the likelyhood of the presence of a specific protein in said sample; and
respectively assigning the observed mass spectrum to a protein of said proteome, based on said scores, thereby identifying the presence of said protein in said sample.

As described herein, mass spectrometry refers to a spectroscopic technique of measuring the mass-to-charge ratio of ions to identify and quantify molecules in simple and complex mixtures.

In particular embodiments, the characterization procedure comprises the steps of collecting the spectrum (1), and spectral matching (2) so as to obtain an spectral assignment (3). In the spectrum collection step, an observed mass spectrum of a peptide is obtained. For the spectral matching, theoretical fragment ion spectra (5) are obtained from a protein database (4). The observed mass spectrum is compared to theoretical fragment ion spectra (5) in a spectral matching step (2). After the spectral matching step (2), the observed mass spectrum is assigned to a protein sequence. By repeating the above procedure for various observed mass spectra, proteins are sequenced.

Preferably, the theoretical fragment ion spectra comprise all fragment ions which might be created from a genome, glycome or proteome in a specific mass spectrometry fragmentation. However, the theoretical fragment ion spectra might comprise more theoretical fragment ions as well. For example, when the method is applied to the study of proteins, the theoretical fragment ion spectra might comprise all a-,b-,c-,x-,y-, and z-series of the target proteome even though it is known that the fragmentation technique used to obtain the observed fragment ion spectrum only produces b- and y-ions. Also, the theoretical fragment ion spectra may not comprise all fragment ions which occur in the sample. For example, the sample may comprise protein fragments comprising post-translational modifications and/or mutations which do not occur in the theoretical fragment ion spectra.

Accordingly, in particular embodiments said theoretical fragment ion spectra are respectively obtained by assuming at least 25% of all possible ions, more preferably at least 75% of all possible ions, most preferably by assuming about all possible ions generated from the protein sequences in said proteome; or by assuming at least 25% of all possible ions, more preferably at least 75% of all possible ions, most preferably by assuming all possible ions generated from the gene sequences in said genome. For amino acids, the recitation “all possible ions” as used herein refers to a set of ions which may comprise all possible ion fragments starting from the o-terminus and all possible ion fragments starting from the n-terminus. For proteins, the “all possible ions” may comprise all a- and x-fragments, all b- and y-fragments, and/or all c- and z-fragments. For proteins, the mass differences between different ions comprised in the “all possible ions” are preferably equal to the mass of an integer number of amino acid residues. For nucleic acids, the “all possible ions” may comprise all ion fragments starting from the 3′ end and starting from the 5′ end. For nucleic acids, the mass differences between different ions comprised in the “all possible ions” are preferably equal to the mass of an integer number of nucleic acid residues.

The methods provided herein thus involve the analysis of a mass spectrum obtained from a sample. Methods of mass spectrometry (MS) and tools for performing them are known in the art and include collision-induced dissociation (CID), also known as “collisionally activated dissociation” (CAD). The method induces fragments of molecular ions in the gas phase. The molecular ions are accelerated and allowed to collide with neutral molecules (such as helium, nitrogen or argon). In the collision some of the kinetic energy is converted into internal energy which results in bond breakage and the fragmentation of the molecular ion into smaller fragments. These fragment ions can then be analyzed by a tandem mass spectrometry. Examples of mass spectrometers include Triple quadrupole mass spectrometers, Fourier transform ion cyclotron resonance, Sustained off-resonance irradiation collision-induced dissociation (SORI-CID) spectrometers and higher-energy collisional dissociation (HCD) or “orbitrap” mass spectrometers. The methods provided herein may include the step of submitting the sample to mass spectrometry but typically start out from the resulting spectrum which is obtained. This is referred to herein as the “query spectrum S” or “S”. In particular embodiments the method is a computer-implemented method.

The sample on which the mass spectrometry analysis is performed is not critical to the methods provided herein. The sample submitted for MS analysis may be solid, liquid, or gas. In light of the analysis method however, the biomacromolecule in the sample can typically be attributed to an organism (also referred to as the target organism herein). When the biomacromolecule is a protein, this allows for the comparison with the proteome of said organism in the methods provided herein.

The methods provided herein may involve a pre-processing step wherein theoretical fragment ion spectra are obtained by assuming at least 25% of all possible ions, more preferably at least 75% of all possible ions, most preferably by assuming all possible ions in the genome, proteome, or glycome of the target organism, depending on whether proteins, nucleic acids, or polysaccharides are investigated.

In particular for proteins, the methods provided herein may involve a pre-processing step wherein the complete a-, b, or c- and respectively x- y- or z-series for every protein in the proteome of the organism of interest is calculated. This step will be required only once for a given organism and can in most cases be ensured based on information retrieved from Genbank (http://www.ncbi.nlm.nih.gov/genbank/) or other publicly accessible sequence databases. The data obtained can then be distributed across the nodes of a multiprocessor or across the cores of a multi-core chipset for a trivial speedup (so called embarrassingly simple parallelization).

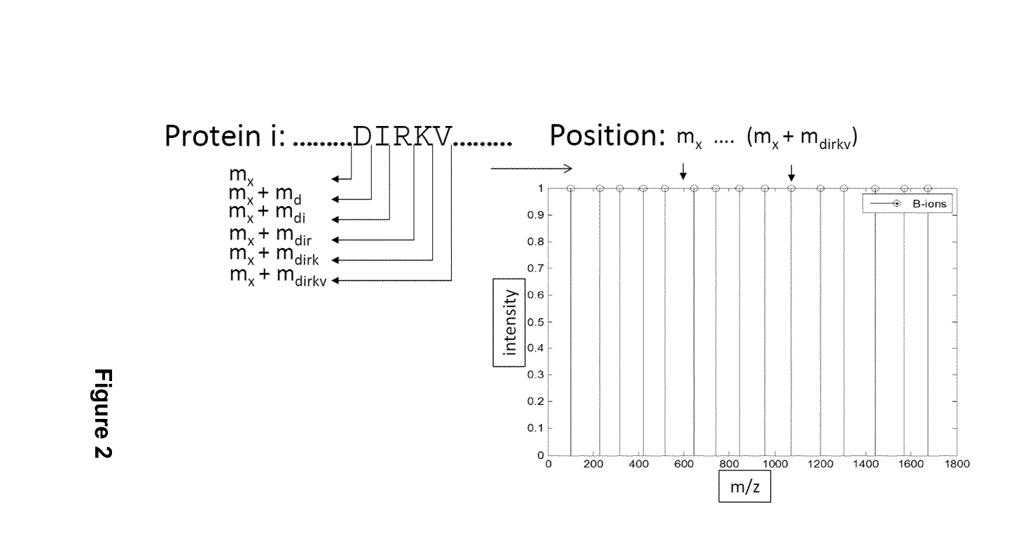

Indeed, for proteins and in order to allow comparison with a query spectrum, the proteome may be converted into a synthetic spectrum by assuming all possible a-, b, or c- and respectively x- y- or z-ions that can be generated from these proteins. Each of the protein sequences in the proteome are converted into a list of pseudo-masses. In this regard, a particular pseudo mass need not correspond to a specific observed ion-value, but its value relative to the other pseudo-masses implies its unique position in the protein sequence. In doing so, a theoretic spectrum is produced in silico. This is illustrated in FIG. 2.

More particularly for proteins, the theoretical fragment ion spectra may be obtained by assuming all possible a-, b-, or c-, and/or x-, y-, or z-generated from the protein sequences in said proteome. The use of all possible a-, b-, or c-, and/or x-, y-, or z-independent of certain assumptions of digestion of the protein in the sample allows for a more accurate analysis which can take into account possible (unknown) modifications of the protein.

Accordingly, in the theoretical fragment ion spectra each of the proteins of the proteome is associated with M theoretical a-, b, or c- and/or x- y- or z-. Potentially, the observed fragment ion masses can be produced by any sub-pattern in the protein sequence. Therefore, M possible starting locations in the protein sequence may be considered to search for a co-occurring pattern that explains the fragment ions in the observed peptide spectrum. When the origin of the observed fragment ion masses are unknown, every fragment ion in the spectrum may be considered as a potential product of the protein at a particular starting location.

Methods according to the present disclosure should be contrasted with prior art methods such as the methods by Wilhelm et al (as referenced in the Background section). Wilhelm et al. tries to explain modifications in observed fragment spectra. Doing so they first apply a biclustering approach to group spectra that share a subset of fragments. Second, they will look at particular mass differences between two bicluster groups to screen for a potential shift of the fragment peaks. So Wilhelm et al looks for structure in the data, but does not identify the spectra. Conversely, the present methods involve comparing an observed mass spectrum of a sample with a theoretical fragment ion spectrum. Thus the present methods may entail searching for patterns, or equivalently subsets, or equivalently mass differences, or equivalently item sets, etc., between an observed mass spectrum of a protein fragment in a sample and a theoretical fragment ion spectrum, or in other words a virtual protein fragment spectrum. Both methods have in common that they may be said to look at patterns that are shared between peak lists, i.e., spectral alignment, but the implementation and application is different. For example, to map peptides on proteins, the present methods can be said to involve disconnecting the b- and y-ion series. Such thing is not possible in the application of Wilhelm et al. as they stick with observed fragment data.

In particular embodiments, the methods comprise generating theoretical ion masses for the target proteome at the a-, b, or c-ion bond and the x- y- or z-ion bond respectively, and computing error intervals for every fragment ion mass based on the error tolerance of the mass spectrometry instrumentation. Indeed, in order to ensure that the theoretical masses can be adequately compared to the peaks in the spectra obtained, the potential error margin of the mass spectrometer used can be taken into consideration.

In a next step, the peaks of the query spectrum S may be compared with the theoretical a-, b, or c- and/or respectively x- y- or z-series values. This is preferably done by spectral convolution. Preferably, the user can specify the mass precision to be considered for this mathematical operation. For every query spectrum S and every protein a/x, b/y, or c/z-series, the m/z value of every query peak from every predicted a/x, b/y, or c/z value is substracted. This step can be done separately, or by matching pooled a/x, b/y, or c/z series. This step is also trivially parallelizable. The data can remain in its original floating point representation or it can be rounded. However, care must be take to round well beyond the accuracy of the instrument. In particular embodiments, rounding is pursued only in cases where the resulting speed gains justify the move away from full floating point representation.

The resulting differences may then be sorted by DBSCAN-like Clustering, as described by Martin Ester, Hans-peter Kriegel, Jorg Sander, and Xiaowei Xu, A density-based algorithm for discovering clusters in large spatial databases with noise, 7^{th} International converence on Data Warehousing and Knowledge Discovery, 1996, pp. 226-231. The sorted deltas are then traversed with every delta assumed to be the initiator of a cluster. If the succeeding delta is within 2ϵ of the previous delta (where ϵ is the error tolerance of the instrument in ppm) the succeeding delta is added to the cluster and the delta value of the cluster is updated by a central value of the delta's composing the cluster (mean, median, mode, max, etc.). It is noted that this step is also parallelizable though not trivially. Accordingly, in particular embodiments, the methods comprise generating theoretical ion masses, preferably for a target proteome or genome, and computing error intervals for every fragment ion mass based on the error tolerance of the mass spectrometry instrumentation. This allows grouping theoretical ion masses which are separated by less than the error tolerance.

In a next step, the methods may encompass a significance analysis and biomacromolecule assignment. The convolution step described above produces a clusters that contain a varying number of delta values that correspond to putative peaks “shifted” relative to the predicted total protein spectrum. The significance of the correspondence can be determined using a formal p-value based on an assumed (modified) Poisson distribution that is suited to deal with the count statistics produced by the DBSCAN algorithm. The modification of the distribution is trivial, in that a normal Poisson distribution entails “zero counts” as a possibility, while in the present methods there are no zero counts as a peak is always matched between the virtual protein and observed peptide peptides. For this reason, the “one counts” are inflated.

The peaks participating in delta clusters considered significant are then assigned to the relevant biomacromolecule entry.

Where the spectrum is from a mixed sample (unintentionally, e.g., chimeric spectra or by design, e.g., in the data-independent acquisition (DIA) experimental paradigm), different subsets of the peaks in the spectrum will be assigned to different biomacromolecules.

An example of a practical implementation of how these steps of spectral convolution, clustering and assignment may be carried out is detailed below for proteins and DNA.

First, a theoretical fragment ion spectrum i is selected corresponding to a given protein or chromosome and it is compared to an observed fragment ion spectrum j.

In particular embodiments, the theoretical fragment ion masses are adjusted by assuming a charge state i.e. the “theoretical fragment ion masses” are updated to “(theoretical fragment masses)/z”. However, this latter step is optional.

In particular embodiments, the method then comprises selecting a mass value py from observed fragment spectrum j, selecting a mass value mx from theoretical fragment spectrum i, aligning the observed spectrum to the theoretical spectrum by computing a mass shift τxy=mx−py and adjusting the observed fragment ion masses by adding τxy, such that the new mass of peak py now equals mx. This can be interpreted as searching for patterns between observed and theoretical data that can be aligned for the annotation of observed fragment ions. In this context, we note that Wilhelm et al (referred to in the background section) looks for mass differences to find patterns that explain modifications. Both the present methods and the disclosure of Wilhelm et al use the terminology of a mass shift, but the interpretation is different. The term “mass shift” is commodity in mass spectrometry.

In a next step the methods may comprise searching for a pattern and scoring it. In particular embodiments this involves:
Computing the number of fragment ion masses that coincide with the adjusted masses of the observed fragment ion spectrum given precomputed error tolerance intervals (count_{xy}); and
Computing the sum of the intensities of the observed fragment ion masses coinciding with theoretical fragment ion masses (sum_{xy}).

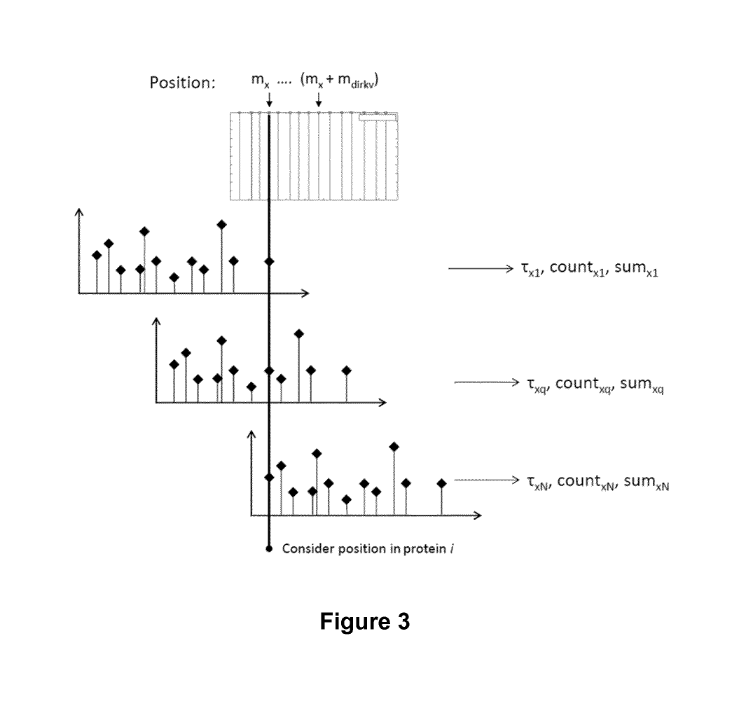

This is repeated for every possible theoretical position in every possible biomacromolecule of the set of reference biomacromolecules. For proteins, the set of reference biomacromolecules corresponds to the reference proteome (FIG. 3). Analogously, for DNA, the set of reference biomacromolecules corresponds to the reference genome.

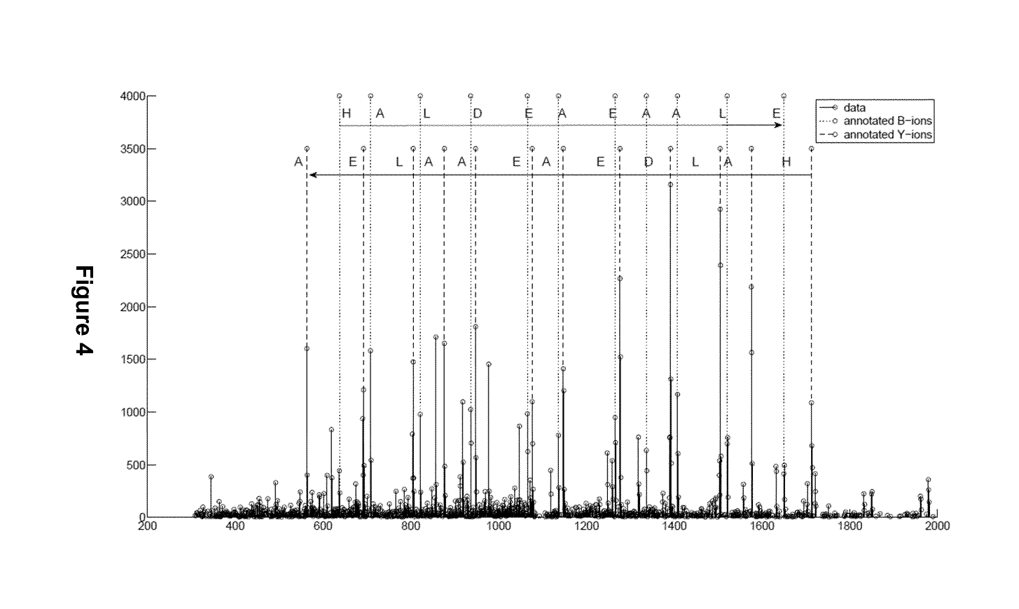

The distribution of the number of coinciding fragment ions is then preferably modeled by a Poisson model whereby for each location a p-value is generated, the p-value representing the probability of a match between an observed fragment ion spectrum and a (part of) a theoretical ion spectrum. Optionally, the local score is correlated for additional confidence. In particular embodiments, positions with a p-value smaller than a pre-determined significance level (for example a significance level of 0.05) are regarded as statistically significant. Thereafter the observed fragment spectrum can be annotated to show which peaks are matched by the theoretical ion fragments (shown for proteins in FIG. 4a). Similarly, the sequence can be updated to show which subsequence has observed ion fragments (shown for proteins in FIG. 4b).

The above process can be repeated for every combination of observed ion spectra and theoretical fragment spectra. This makes it possible to identify the presence of a given biomacromolecule, for example a given protein, in the sample. In particular embodiments, the sample comprises more than one biomacromolecule from an organism. In particular embodiments, the one or more biomacromolecules are proteins or nucleic acids. In particular embodiments, the one or more biomacromolecules are proteins.

The application of the methods provided herein are numerous. Indeed, the specific advantages of the methods provided herein allow for analysis of sets of biomacromolecules, for example for analysis of the proteome, in conditions where prior art methods have failed, due to limitations on the nature of the sample and/or the need for prior knowledge of the starting material. The following are exemplary applications of the provided methods.

Post Translational Modifications/Mutations (PTM): while different approaches to the problem of proteins comprising PTM have been published, the present methods broaden the applicability of PTM/Mutation tolerant search by eliminating the requirement for an enzymatically-constrained peptide population. In addition, by avoiding the reliance on a complementary reference search, the present methods preserve the ability to provide straightforward p-values since they do not involve complex, difficult to analyze iterative searches such as employed by many “tolerant” search techniques (which are usually required to eschew probabilistic characterization of their results). Accordingly, in particular embodiments, the methods are performed to identify proteins which may be subject to PTM.

Endogenous Peptidomics: Since the methods provided herein do not require prior knowledge of the process which generated the peptide population being analyzed, they are ideal for endogenous peptidomics. Endogenous peptides are often modified in-vivo prior to full biological activation. Since the methods provided herein do not require prior knowledge of these modifications they have the unique advantage of their ability to detect this challenging peptide population. Accordingly, in particular embodiments, the methods are performed to identify endogenous peptides.

Searching Chimeric Spectra: Chimeric spectra represent another challenging case for typical search engines (which usually address them through an iterative scheme which, as previously mentioned, causes difficulty in the calculation of valid p-values). The present methods, by not requiring pure spectra, provide a natural match for such spectra. Accordingly, in particular embodiments, the methods are performed to identify biomacromolecules present in a sample generating an chimeric spectrum.

Data Independent Acquisition: A particularly competitive match for the methods and systems according to the present invention is the analysis of DIA-based spectra. These are well-known for their complexity since multiple precursors are fragmented concurrently. The present methods will do particularly well in this situation since it does not require knowledge of the precursors (i.e. there is no need for the low-energy scan—in the case of Waters® data—or the information about the SWATH™ window in the case of ABSciex® data).

Label Free (fragment-based) Quantification: DIA-based quantitative proteomics has been an appealing prospect thanks to the freedom from the stochasticity of precursor selection. However, the difficulty in the identification of the resulting spectra has limited its spread. By potentially eliminating the need for precursor information while still maintaining excellent identifiability, the present methods enable fragment-based quantification on much cheaper instrumentation then is currently feasible. This allows quantitative proteomics to be performed on devices capable of accurate mass but limited to all-ion fragmentation (hence much cheaper than the typical instruments used for quantitative proteomics).

Spectral library matching: In spectral library matching an unknown observed peptide spectrum is queried against a library of high quality fragment spectra for which the peptide sequence assignment is known. In the approach according to the present invention, spectral library searching is tolerant against unspecified PTMs.

DNA modifications: The present invention can be used to search other biological sequences as well. Short DNA sequences can be fragmented using tandem mass spectrometry. The resulting fragment ions are organized in a similar way as peptides. The present invention can be used to map the fragment ions onto the genome sequence. Since, methods according to the present invention can handle unspecified modification this technique is suited to understand DNA methylation and other epigenetic signaling.

Invariant with respect to the fragmentation principle: Methods according to the present invention are invariant of the type of fragmentation and works equally well for collision-induced dissociation (CID), electron-capture dissociation (ECD), electron-transfer dissociation (ETD), negative electron-transfer dissociation (NETD), electron-detachment dissociation (EDD), photodissociation, particularly infrared multiphoton dissociation (IRMPD) and blackbody infrared radiative dissociation (BIRD), surface-induced dissociation (SID), Higher-energy C-trap dissociation (HCD), charge remote fragmentation. For these applications it is not required to define new fragment ion series, since the mass difference between consecutive fragment ions remains fixed for all ion fragment types.

In particular embodiments, the methods provided herein are computer-implemented methods.

Accordingly, in some embodiments, the application provides computer-implemented methods for determining a biomacromolecule of an organism in a sample, the method comprising the steps of:
a. receiving a biomacromolecule database comprising the biomacromolecule sequences, for example of the genome or of the proteome, of said organism;
b. determining theoretical fragment ion spectra for the protein sequences, the theoretical fragment ion spectra comprising M theoretical fragment ion masses, the theoretical fragment ion spectra corresponding to biomacromolecules i;
c. receiving an observed sample spectrum corresponding, the observed spectrum comprising N observed fragment ion masses;
d. matching the observed sample spectrum to the theoretical fragment ion spectrum or a part thereof by clustered spectral convolution; and
e. identifying a protein present in said sample based thereon.

In particular embodiments, the methods are designed to take into consideration the error tolerance of the mass spectrometry method. Accordingly, in particular embodiments, the computer-implemented methods further comprise the step of receiving an error tolerance, which can then be taken into consideration in the methods as described herein.

Further provided herein is a computer readable medium having stored thereon instructions which when executed by a computing device or system cause the computing device or system to perform the steps of a method provided herein.

The application further provides data-processing systems comprising means configured for carrying out the methods provided herein.

The application further provides computer program products having instructions which when executed by a computing device or system cause the computing device or system to perform a method as described herein.

The application further provides data streams which are representative of a computer program having instructions which when executed by a computing device or system cause the computing device or system to perform the methods as described herein.

### EXAMPLES

1. Identification of a Protein in a CID Spectrum

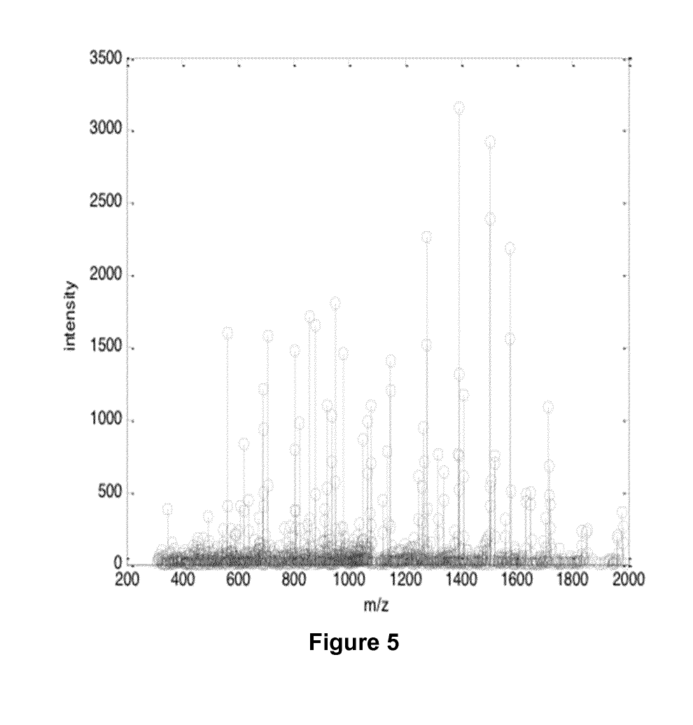

The CID spectrum in FIG. 5 is searched against a protein with the sequence:

(SEQ ID NO: 1)
IIDEWRKKTDDLAAELDGAQRDLRNTSTDLFKAKNAQEELAEVVEGL
RRENKSLSQEIKDLTDQLGEGGRSVHEMQKIIRRLEIEKFELQHAL
DEAEAALEAEESKVLRAQVEVSQIRSEIEKRIQEKFEEFENTRKNH
ARALESMQASLE

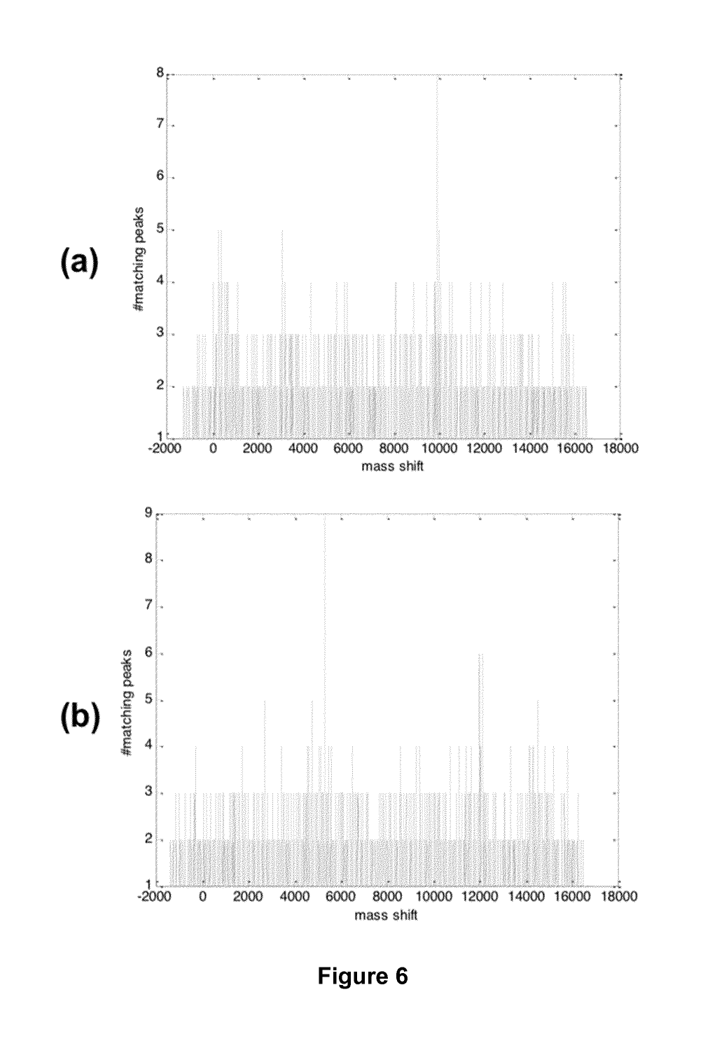

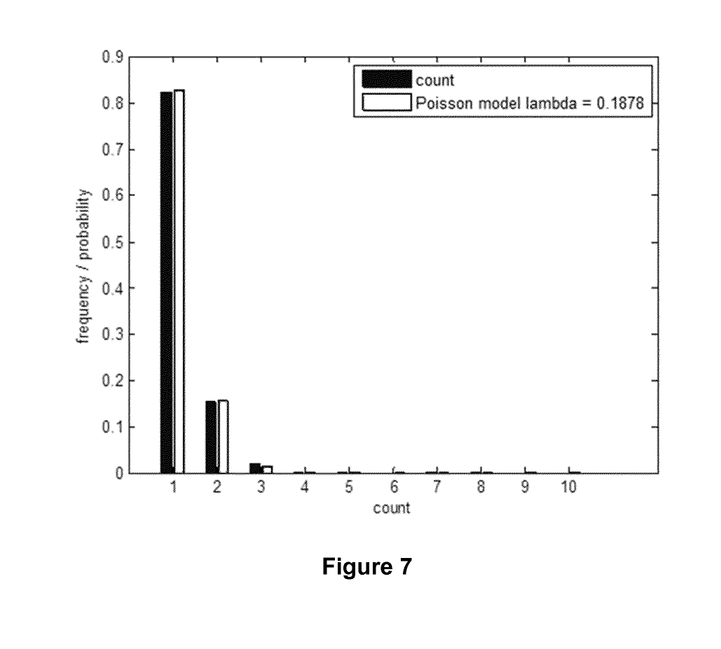

Applying the methods according to the present invention with an error tolerance of 0.05 Da yields b-ion (1+) and y-ion (1+) matching pseudo-ion counts as seen in FIGS. 6a and 6b. The count score is modeled by a Poisson distribution (see FIG. 7) and p-values are generated for each count. Counts of 8 and 9 as are seen in the y-ion and b-ion series respectively are statistically significant and lead to the following annotation:

Result in terms of sequence:

1. B-ion result: LQHALDEAEAALEAEE [p-val: 6.8e-13] (SEQ ID NO: 2)

2. Y-ion result: LQHALDEAEAALEAEE [p-val: 3.3e-11] (SEQ ID NO: 3) are confirmed by the SEQUEST algorithm as: EELQHALDEAEAALEAEESK (SEQ ID NO:4)

2. Exemplary Workflow

The methods provided herein are applied to protein characterization, as schematically illustrated in FIG. 1. In particular, the characterization procedure comprises the step of spectrum collection (1). In the spectrum collection step, an observed mass spectrum of a peptide is obtained. The observed mass spectrum is compared to theoretical fragment ion spectra (5) in a spectral matching step (2). Prior to the spectral matching step (2), the theoretical fragment ion spectra (5) were derived from a protein database (4). After the spectral matching step (2), the observed mass spectrum is assigned to a protein sequence. By repeating the above procedure for various observed mass spectra, proteins are sequenced. The sequence coverage for a specific protein I (6) is schematically shown at the lower end of the figure.

While this invention has been described in conjunction with the specific embodiments outlined above, it is evident that many alternatives, modifications, and variations will be apparent to those skilled in the art. Accordingly, the preferred embodiments of the invention as set forth above are intended to be illustrative, not limiting. Various changes may be made without departing from the spirit and scope of the inventions as defined in the following claims.

It is noted that citation or identification of any document in this application is not an admission that such document is available as prior art to the present invention.

1. A computer-implemented method for determining the presence of a biomacromolecule of an organism in a sample, said biomacromolecule being a protein or a nucleic acid, the method comprising:
obtaining an observed mass spectrum of the sample from mass spectrometry instrumentation, thereby obtaining a set of query peaks;
subtracting a mass-charge ratio m/z value of every query peak from theoretical fragment ion masses of a target proteome or genome of said organism obtained by determining theoretical fragment ion spectra comprising theoretical fragment ion masses for the protein or nucleic acid sequences of said target proteome or genome, respectively, to obtain differences;
clustering and scoring the resulting differences, thereby obtaining scores representative of the likelyhood of the presence of a specific protein or gene in said sample; and
respectively assigning the observed mass spectrum to a protein of said proteome, or to a gene of said genome, based on said scores, thereby identifying the presence of said protein or gene in said sample.

2. The method according to claim 1;
wherein the biomacromolecule is a protein.

3. The method according to claim 1;
wherein the observed mass spectrum is obtained by tandem mass spectroscopy.

4. The method according to claim 1;
wherein said theoretical fragment ion spectra are respectively obtained by:
assuming that at least 25% of all possible ions are generated from the protein sequences in said proteome; or
assuming that at least 25% of all possible ions are generated from the gene sequences in said genome.

5. The method according to claim 1, further comprising:
generating theoretical ion masses for the target proteome or genome and computing error intervals for every fragment ion mass based on an error tolerance of the mass spectrometry instrumentation.

6. The method according to claim 5, further comprising:
selecting a theoretical fragment ion spectrum i corresponding to a given protein or chromosome and comparing it to an observed fragment ion spectrum j.

7. The method according to claim 6, further comprising:
for every observed fragment mass, selecting a mass value py from the observed fragment ion spectrum j, selecting a mass value mx from the theoretical fragment ion spectrum i, and aligning the observed fragment ion spectrum j to the theoretical fragment ion spectrum j by:
computing a mass shift τxy=mx−py; and
adjusting the observed fragment ion masses by adding τxy, such that the new mass of peak py now equals mx.

8. The method according to claim 7, further comprising:
searching for a pattern and scoring said pattern by a method comprising the steps of:
a) computing the number of fragment ion masses that coincide with the adjusted masses of the observed fragment ion spectrum given the precomputed error tolerance intervals (count_{xy}); and
b) computing the sum of the intensities of the observed fragment ion masses coinciding with theoretical fragment ion masses (sum_{xy}).

9. The method of claim 8, further comprising:
modeling a distribution of the number of coinciding fragment ions by a Poisson model and generating for each location a p-value for a probability of a match between an observed fragment ion spectrum and a part of a theoretical ion spectrum, and optionally correlating a local score distribution for additional confidence, wherein positions with a p-value smaller than a pre-determined significance level are regarded as statistically significant.

10. The method of claim 8, further comprising:
annotating the observed fragment ion spectrum j to show which peaks were matched by the theoretical ion fragments and updating the sequence to show which subsequence had observed matching ion fragments.

11. The method according to claim 1;
wherein the theoretical fragment ion masses are adjusted by assuming a charge state z:
the “theoretical fragment ion masses” are updated to “(theoretical fragment masses)/z”.

12. The method according to claim 1;
wherein the sample comprises more than one biomacromolecule from said organism.

13. The method according to claim 1;
wherein the biomacromolecule is a nucleic acid.

14. A data-processing system comprising:
a processor; and
a non-transitory computer readable medium having computer readable instructions stored thereon, which, when executed by the processor, configures the processor to perform the method according to claim 1.

15. A non-transitory computer readable medium having computer readable instructions stored thereon, which, when executed by a computing device or system, cause the computing device or system to perform the method according to claim 1.
